(19) United States
(12) Patent Application Publication
(10) Pub. No.: US 2011/0112041 A1
(43) Pub. Date: May 12, 2011

(54) Treatment of Male-Pattern Baldness by Local Induction of the Metabolic Defect of Fabry Disease

(75) Inventor: Raphael Schiffmann, Rockwall, TX (US)

(73) Assignee: Baylor Research Institute, Dallas, TX (US)

(21) Appl. No.: 12/896,591

(22) Filed: Oct. 1, 2010

Related U.S. Application Data
(60) Provisional application No. 61/247,894, filed on Oct. 1, 2009.

(51) Int. Cl. A61K 31/7004; A61P 17/14

(52) U.S. Cl. 514/23

(57) ABSTRACT

Compositions comprising inhibitors of lysosomal enzyme alpha-galactosidase A and their use to favor hair growth or to prevent hair loss are disclosed in the instant invention. The topical use of the composition of the instant invention directly inhibits scalp enzyme activity and is used in the treatment for male pattern baldness (androgenic alopecia) in men and women.

### CROSS-REFERENCE TO RELATED APPLICATIONS

This application claims priority to U.S. Provisional Application Ser. No. 61/247,894, filed Oct. 1, 2009, the entire contents of which are incorporated herein by reference

### TECHNICAL FIELD OF THE INVENTION

The present invention relates in general to the field of male-pattern baldness treatment agents, and more particularly to the topical use of an inhibitor of the lysosomal enzyme alpha-galactosidase A to favor hair growth or to prevent hair loss.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with the use of topical agents to inhibit scalp enzyme activity in order to favor hair growth or to prevent hair loss.

U.S. Pat. No. H1,551 issued to Wakoh (1996) describes a topically applicable novel hair grower that does not exert undesirable side-effects. The hair grower contains a maximum of about 2% of oxendolone in combination with a topically applicable vehicle or carrier, if desired, with further conventional additives. The composition of the Wakoh patent inhibits both the activity of the reductase and the binding of 5α-DHT with the receptor protein by topical use.

U.S. Pat. No. 6,497,892 issued to Pugliese and Pugliese (2002) discloses a hair and scalp formulation based on a peroxidase enzyme and synergistic yeast extract therefore. It entails a chemically-inert carrier material, a germicidal/preservative agent, and a metal chelating agent. The '892 patent further describes an aqueous formulation adapted for topical hair rinsing consisting of a peroxidase enzyme, a yeast extract adapted to enhance the activity of the enzyme, and a cationic hair conditioning agent selected from water-soluble thickening resins.

U.S. Pat. No. 7,556,825 issued to Li and Lishko (2009) describes a method for targeted and specific delivery of beneficial compounds, including hair dyes, melanin, proteins, and nucleic acids for gene therapy, to hair follicle cells using liposomes encapsulating the beneficial compound. Particularly preferred methods describe delivery of hair dyes, melanin or tyrosinase to the hair follicle for the purpose of improving hair color or condition, the delivery of compounds which prevent alopecia or stimulate hair growth, either by encapsulating a compound in liposomes or by encapsulating a nucleic acid capable of expressing a protein in liposomes. Also described are liposome compositions for practicing the methods.

### SUMMARY OF THE INVENTION

The present invention describes the use of inhibitor of the lysosomal enzyme alpha-galactosidase A to directly inhibit scalp enzyme activity in order to favor hair growth or to prevent hair loss as a treatment for male pattern baldness (androgenic alopecia) in men and women.

In one embodiment the present invention discloses a topical pharmaceutical formulation for stimulating hair growth, preventing hair loss, treating baldness and alopecia in a human subject comprising: one or more inhibitors of lysosomal α-galactosidase A, wherein the one or more inhibitors are dispersed or dissolved in a continuous phase, wherein the continuous phase is an aqueous phase, an organic phase or an oily phase and one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. In one aspect of the present invention the one or more inhibitors of lysosomal α-galactosidase A comprise 1-deoxygalactonojirimycin salts and derivatives, iminosugars and derivatives, deoxyazasugars and derivatives, calystegines alkaloids and derivatives, fagomine isomers, conduritol C epoxides, nortropane alkaloids or any combinations thereof. In a specific aspect the inhibitor of the lysosomal α-galactosidase A is 1-deoxygalactonojirimycin hydrochloride present in an amount of about 1 to 15% by weight in the composition. In another aspect the composition is applied to a hair scalp once daily or multiple times in a day. In yet another aspect the composition stimulates hair growth, prevents hair loss, treats baldness and alopecia by inhibiting the activity of one or more scalp enzymes and is applied as a topical pharmaceutical formulation in the form of a solution, a suspension, an ointment, a foam, an emulsion or a cream.

In related aspects the emollient comprises glycerin, propylene glycol, polyethylene glycol or any combinations thereof, the thickener comprises amides, cellulose, vinylpyrrolidone polymers, carboxypolymethylene or any combinations thereof, the bacteriostatic agents comprise benzoic acid and its esters, benzilic alcohol, parabens or any combinations thereof, the propellants comprise propane, butane, a mixture of propane and butane or any combinations thereof, the surfactants and emulsifiers comprise laurylsulphate, sorbitane esters and ethers, polyoxymethylene esters and ethers, polyoxyethylene-sorbitane esters and ethers or any combinations thereof.

The hair loss, the baldness or the alopecia treated by the composition of the present invention are caused by one or more factors selected from the group consisting of age, genetic make up, environmental effects, chemotherapy, childbirth, fertility drugs, major surgery, poisoning, severe stress, iron deficiency, radiation, mycotic infections, autoimmune disorders, lupus, tumors, skin outgrowths, hypothyroidism, and hyperthyroidism.

In another embodiment the present invention describes a method of stimulating hair growth, preventing hair loss or both in a subject comprising the steps of: identifying a subject in need of stimulation or hair growth, prevention of hair loss or both and applying a therapeutically effective amount of a topical pharmaceutical formulation comprising one or more inhibitors of lysosomal α-galactosidase A to a hair scalp of the subject once or multiple times daily, wherein the one or more inhibitors are dispersed or dissolved in a continuous phase, wherein the continuous phase is an aqueous phase, an organic phase or an oily phase. The method further comprises the step of monitoring the subject for increased hair growth or decreased hair loss following the application of the topical pharmaceutical formulation for a specified period of time. The topical pharmaceutical formulation of the method of the present invention further comprises one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. The topical pharmaceutical formulation is a solution, a suspension, an ointment, a foam, an emulsion or a cream and stimulates hair growth, prevents hair loss or both by inhibiting the activity of one or more scalp enzymes. In a specific aspect the inhibitor of the lysosomal α-galactosidase A is 1-deoxygalactonojirimycin hydrochloride.

In yet another embodiment the present invention describes a method of treating baldness or alopecia in a subject comprising the steps of: identifying the subject in need of treatment against the baldness or the alopecia and applying a therapeutically effective amount of a topical pharmaceutical formulation comprising one or more inhibitors of lysosomal α-galactosidase A to a hair scalp of the subject once or multiple times a day, wherein the one or more inhibitors are dispersed or dissolved in a continuous phase, wherein the continuous phase is an aqueous phase, an organic phase or an oily phase.

The method of the present invention further comprises the step of monitoring the subject for hair growth following the application of the topical pharmaceutical formulation for a specified period of time, wherein the topical pharmaceutical formulation comprises one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. The topical pharmaceutical formulation of the method of the present invention is a solution, a suspension, an ointment, a foam, an emulsion or a cream and promotes hair growth by inhibiting the activity of the one or more scalp enzymes.

In one aspect the alopecia comprises androgenic alopecia, traction alopecia, alopecia areata, alopecia totalis, alopecia universalis, alopecia mucinosa or any combinations thereof. In specific aspects of the method the inhibitor of the lysosomal α-galactosidase A is 1-deoxygalactonojirimycin hydrochloride present in an amount of about 1 to 15% by weight in the composition.

In various embodiments the present invention describes formulations of the 1-deoxygalactonojirimycin hydrochloride used as the inhibitor of the lysosomal α-galactosidase A. In one embodiment the present invention discloses a topical cream or an emulsion for stimulating hair growth, preventing hair loss, treating baldness and alopecia in a human subject comprising: a 1-deoxygalactonojirimycin hydrochloride solution in purified water or a buffer, wherein the amount of the 1-deoxygalactonojirimycin hydrochloride is 1 to 15% by weight, and one or more of the listed excipients: (i) an oil phase selected from the group consisting of solid paraffin, liquid paraffin, silicone oil, squalane, glyceryl monooleate, olive oil, a higher alcohol, a higher aliphatic acid and isopropyl myristate, wherein the oil phase is emulsified with the 1-deoxygalactonojirimycin hydrochloride solution, (ii) an emollient selected from the group consisting of glycerin, propylene glycol, and polyethylene glycol, (iii) a thickener selected from the group consisting of amides, cellulose, vinylpyrrolidone polymers, carboxypolymethylene, (iv) a surfactant selected from the group consisting of laurylsulphate, sorbitane esters and ethers, polyoxymethylene esters and ethers, polyoxyethylene-sorbitane esters and ethers, and (v) a bacteriostatic agent selected from the group consisting of benzoic acid and its esters, benzilic alcohol, and parabens. In one aspect the composition stimulates hair growth, prevents hair loss, and treats baldness and alopecia by inhibiting the activity of the one or more scalp enzymes. In another aspect the composition is applied to a hair scalp twice daily with an applicator or is rubbed in with one or more fingers.

In another embodiment the present invention discloses a pharmaceutical foam for stimulating hair growth, preventing hair loss, treating baldness and alopecia in a human subject comprising: a 1-deoxygalactonojirimycin hydrochloride solution in purified water or a buffer, wherein the amount of the 1-deoxygalactonojirimycin hydrochloride is 1 to 15% by weight and one or more inactive ingredients selected from the group consisting of butane, butylated hydroxytoluene, cetyl alcohol, citric acid, glycerin, propylene glycol, polyethylene glycol isobutane, lactic acid, amides, cellulose, vinylpyrrolidone polymers, carboxypolymethylene polysorbate 60, propane, ethyl alcohol, and stearyl alcohol. In one aspect the composition stimulates hair growth, prevents hair loss, treats baldness and alopecia by inhibiting the activity of the one or more scalp enzymes and is applied to a hair scalp twice daily by rubbing into the hair scalp with one or more fingers.

In yet another embodiment the present invention discloses a topical pharmaceutical solution for stimulating hair growth, preventing hair loss, treating baldness and alopecia in a human subject comprising: a 1-15% by weight solution of 1-deoxygalactonojirimycin hydrochloride in purified water, ethyl alcohol, propylene glycol, and a bacteriostatic agent selected from the group consisting of benzoic acid and its esters, benzilic alcohol, and parabens. The topical pharmaceutical solution of the present invention is applied to a hair scalp twice daily with an applicator.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It is to be understood that as used in the present application, the term “baldness” is intended to be any degree of thinning of the hair. The term “androgenic alopecia” refers to an autosomal disorder which begins in puberty in genetically disposed individuals. Androgenic alopecia is also known as hereditary baldness, male pattern baldness, and seborrheic alopecia. Androgenic alopecia may occur in males and females.

As used herein, the term “lysosomal enzyme” as used herein is construed to comprise enzymes present in the lysosome and other enzymes intervening in lysosomal biogenesis. Some of the most commonly known lysosomal enzymes in the art include β-glucosidase (beta-glucocerebrosidase), β-hexosaminidase, β subunit, β-hexosaminidase, α subunit, β-D-mannosidase, α-L-fucosidase, Arylsulphatase B, Arylsulphatase A, α-N-acetylgalactosaminidase, Aspartylglucosaminidase, Iduronate-2-sulfatase, Glucosamine-N-sulfatase, α-N-acetylglucosaminidase, AcetylcoenzymeA: α-glucosaminide-N-acetyltransferase, N-acetylglucosamine-6-sulfatase, acetylgalactosamine-6-sulfatase, β-D-glucuronidase, Hyaluronidase, Arylsulphatase A, B, C, α-L-mannosidase, α-neuraminidase, Steroid sulfatase, Phosphotransferase, Acid lipase, Tryoleil lipase, Cholesteryl esterase, Acid ceramidase, Sphingomyelinase, α-glucosidase, Palmitoyl protein, thioesterase, Carboxipeptidase, Lysosomal membrane protein, β-galactocerebrosidase, and Acid phosphatase.

The term “pharmaceutically acceptable excipients” as used herein refers to any physiologically inert, pharmacologically inactive material known to one skilled in the art, which is compatible with the physical and chemical characteristics of the active ingredient selected for use. Pharmaceutically-acceptable excipients include, but are not limited to, polymers, resins, plasticizers, fillers, binders, lubricants, glidants, disintegrants, solvents, co-solvents, buffer systems, surfactants, preservatives, sweetening agents, flavoring agents, pharmaceutical grade dyes or pigments, and viscosity agents.

The term “bacteriostatic agent” as used in this invention is synonymous to and may be interchangeable with bacteriocide, germicide, fungicide, disinfectant and other terms commonly used to indicate growth control, either by killing or suppressing, of various bacteria and germs.

The term “propellant” as used in the present invention includes those compounds known in the aerosol art which are either liquefied normally gaseous substances or mixtures of liquefied normally gaseous substances and low boiling liquids, which exhibit a vapor pressure sufficiently high to propel a liquid from a pressurized aerosol container.

The term “surfactant” as used herein refers to compounds which reduce the surface tension of water when dissolved in water or aqueous solutions, or which reduce interfacial tension between two normally immiscible liquids (e.g., oil and water).

The term “suspension” as used herein broadly refers of a mixture having both solid and liquid components where the solid components are not all dissolved in the liquid components. The term “ointment” as used herein refers to formulations (including creams) having oleaginous, absorption, water-soluble and emulsion-type bases, e.g., petrolatum, lanolin, polyethylene glycols, as well as mixtures thereof. As used herein the term “foam” is intended to denote a mixture of liquid and air, the air having been mechanically combined with the liquid as opposed to being chemically generated in situ.

The term “topical use or application” is used herein to refer to local administration of the composition and its various embodiments, for example, in the treatment of alopecia. The composition according to the present invention can be in the form of solutions, lotions, salves, creams, ointments, liposomes, sprays, gels, roller sticks or any other method using micelles and pharmaceutically acceptable penetration enhancers.

The term “chemotherapy” as used herein refers to the treatment of cancer using specific chemical agents or drugs that are destructive of malignant cells and tissues. Also, “chemotherapy” refers to the treatment of disease using chemical agents or drugs that are toxic to the causative agent of the disease, such as a virus, bacterium, or other microorganism. The term “tumor” refers to any new and abnormal cell growth, specifically a new growth of tissue which is uncontrolled and progressive.

The term “autoimmune disorder” as used in the present invention is an umbrella-like term which is used to refer to a large group of unrelated illnesses with unknown causes, but associated with abnormalities in immunoregulation.

The terms “effective amount” or “therapeutically effective amount” refers to the amount of the compound that will elicit the biological or medical response of a tissue, system, animal or human that is being sought by the researcher, veterinarian, medical doctor or other clinician.

As used herein, the term “treatment ” or “treating” refers to any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The present invention describes the topical use of inhibitors of the lysosomal enzyme alpha-galactosidase A to directly inhibit scalp enzyme activity in order to favor hair growth or to prevent hair loss as a treatment for male pattern baldness (androgenic alopecia) in men and women.

Fabry disease is an X-linked disorder of glycosphingolipids that is caused by the deficiency of α-galactosidase A.¹ Data from over 200 male patients treated by the inventor reveals that patients with the classic form of the Fabry's disease do not develop male pattern baldness as part of their dysmorphic characteristics.² More than half of men are affected by male pattern baldness by age 50, and baldness treatments are estimated to be a US $1 billion per year industry. Many women are also affected. Therefore, local induction of the metabolic defect of Fabry disease is likely to reproduce the effect of the disease and maintain normal hair.

The present inventor has observed the absence of male pattern baldness (androgenic alopecia) in patients with Fabry disease and little or no alpha-galactosidase A activity.

Current methods use anti-androgenic activity including Finasteride (Propecia), ketoconazole, based on irradiation, hair transplantion, laser or diet and lifestyle changes or unknown mechanisms such as minoxidil.³ The idea to inhibit alpha-galactosidase A to promote hair growth in otherwise healthy subjects as described herein is completely novel.

The present invention focuses on: (i) topical use of inhibitors of alpha-galactosidase A to effectively make the skin, including the hair follicle deficient in this enzyme activity thus reproducing locally the effect of the systemic disease. Such treatment will prevent or reverse male pattern baldness (androgenic alopecia) and (ii) use of other molecules that reproduce the downstream effect of alpha-galactosidase A deficiency.

Fabry disease is an X-linked disorder of glycosphingolipids that is caused by the deficiency of α-galctosidase A. Fabry disease is a risk factor for developing a systemic vasculopathy, progressive kidney disease, many cardiac complications and ischemic stroke. Previous research by the present inventors has indicated that in patients afflicted with Fabry's disease there is an increased production of non-nitric oxide (NO) reactive species (such as superoxide) especially in blood vessel wall and vascular endothelial cells that likely results from a dysfunction of endothelial nitric oxide synthase (eNOS). BH4 is an essential factor for normal eNOS activity. In the event of BH4 deficiency, eNOS switches from producing NO to generating oxygen free radicals that increase oxidant stress in blood vessels leading to vascular damage.

In one embodiment the present invention discloses a topical pharmaceutical formulation for stimulating hair growth, preventing hair loss, treating baldness and alopecia in a human subject comprising: one or more inhibitors of lysosomal α-galactosidase A, wherein the one or more inhibitors are dispersed or dissolved in a continuous phase, wherein the continuous phase is an aqueous phase, an organic phase or an oily phase and one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. In one aspect of the present invention the one or more inhibitors of lysosomal α-galactosidase A comprise 1-deoxygalactonojirimycin salts and derivatives, iminosugars and derivatives, deoxyazasugars and derivatives, calystegines alkaloids and derivatives, fagomine isomers, conduritol C epoxides, nortropane alkaloids or any combinations thereof. In a specific aspect the inhibitor of the lysosomal α-galactosidase A is 1-deoxygalactonojirimycin hydrochloride present in an amount of about 1 to 15% by weight in the composition. In another aspect the composition is applied to a hair scalp once daily or multiple times in a day. In yet another aspect the composition stimulates hair growth, prevents hair loss, treats baldness and alopecia by inhibiting the activity of one or more scalp enzymes and is applied as a topical pharmaceutical formulation in the form of a solution, a suspension, an ointment, a foam, an emulsion or a cream.

In related aspects the emollient comprises glycerin, propylene glycol, polyethylene glycol or any combinations thereof, the thickener comprises amides, cellulose, vinylpyrrolidone polymers, carboxypolymethylene or any combinations thereof, the bacteriostatic agents comprise benzoic acid and its esters, benzilic alcohol, parabens or any combinations thereof, the propellants comprise propane, butane, a mixture of propane and butane or any combinations thereof, the surfactants and emulsifiers comprise laurylsulphate, sorbitane esters and ethers, polyoxymethylene esters and ethers, polyoxyethylene-sorbitane esters and ethers or any combinations thereof.

The hair loss, the baldness or the alopecia treated by the composition of the present invention are caused by one or more factors selected from the group comprising age, genetic make up, environmental effects, chemotherapy, childbirth, fertility drugs, major surgery, poisoning, severe stress, iron deficiency, radiation, mycotic infections, autoimmune disorders, lupus, tumors, skin outgrowths, hypothyroidism, and hyperthyroidism.

In another embodiment the present invention describes a method of stimulating hair growth, preventing hair loss or both in a subject comprising the steps of: identifying a subject in need of stimulation or hair growth, prevention of hair loss or both and applying a topical pharmaceutical formulation comprising one or more inhibitors of lysosomal α-galactosidase A to a hair scalp of the subject once or multiple times daily, wherein the one or more inhibitors are dispersed or dissolved in a continuous phase, wherein the continuous phase is an aqueous phase, an organic phase or an oily phase. The method further comprises the step of monitoring the subject for increased hair growth or decreased hair loss following the application of the topical pharmaceutical formulation for a specified period of time. The topical pharmaceutical formulation of the method of the present invention further comprises one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. The topical pharmaceutical formulation is a solution, a suspension, an ointment, a foam, an emulsion or a cream and stimulates hair growth, prevents hair loss or both by inhibiting the activity of one or more scalp enzymes. In a specific aspect the inhibitor of the lysosomal α-galactosidase A is 1-deoxygalactonojirimycin hydrochloride.

In yet another embodiment the present invention describes a method of treating baldness or alopecia in a subject comprising the steps of: identifying a subject in need of treatment against the baldness or the alopecia and applying a topical pharmaceutical formulation comprising one or more inhibitors of lysosomal α-galactosidase A to a hair scalp of the subject once or multiple times daily, wherein the one or more inhibitors are dispersed or dissolved in a continuous phase, wherein the continuous phase is an aqueous phase, an organic phase or an oily phase.

The method of the present invention further comprises the step of monitoring the subject for hair growth following the application of the topical pharmaceutical formulation for a specified period of time, wherein the topical pharmaceutical formulation comprises one or more optional pharmaceutically acceptable excipients, wherein the excipients comprise emollients, thickeners, ionic or non-ionic surfactants and emulsifiers, bacteriostatic agents, coloring agents, scents, and propellant gases or any combinations thereof. The topical pharmaceutical formulation of the method of the present invention is a solution, a suspension, an ointment, a foam, an emulsion or a cream and promotes hair growth by inhibiting the activity of the one or more scalp enzymes.

In one aspect the alopecia comprises androgenic alopecia, traction alopecia, alopecia areata, alopecia totalis, alopecia universalis, alopecia mucinosa or any combinations thereof. In specific aspects of the method the inhibitor of the lysosomal α-galactosidase A is 1-deoxygalactonojirimycin hydrochloride present in an amount of about 1 to 15% by weight in the composition.

In various embodiments the present invention describes formulations of the 1-deoxygalactonojirimycin hydrochloride used as the inhibitor of the lysosomal α-galactosidase A. In one embodiment the present invention discloses a topical cream or an emulsion for stimulating hair growth, preventing hair loss, treating baldness and alopecia in a human subject comprising: a 1-deoxygalactonojirimycin hydrochloride solution in purified water or a buffer, wherein the amount of the 1-deoxygalactonojirimycin hydrochloride is 1 to 15% by weight, and one or more of the listed excipients: (i) an oil phase selected from the group consisting of solid paraffin, liquid paraffin, silicone oil, squalane, glyceryl monooleate, olive oil, a higher alcohol, a higher aliphatic acid and isopropyl myristate, wherein the oil phase is emulsified with the 1-deoxygalactonojirimycin hydrochloride solution, (ii) an emollient selected from the group consisting of glycerin, propylene glycol, and polyethylene glycol, (iii) a thickener selected from the group consisting of amides, cellulose, vinylpyrrolidone polymers, carboxypolymethylene, (iv) a surfactant selected from the group consisting of laurylsulphate, sorbitane esters and ethers, polyoxymethylene esters and ethers, polyoxyethylene-sorbitane esters and ethers, and (v) a bacteriostatic agent selected from the group consisting of benzoic acid and its esters, benzilic alcohol, and parabens. In one aspect the composition stimulates hair growth, prevents hair loss, and treats baldness and alopecia by inhibiting the activity of the one or more scalp enzymes. In another aspect the composition is applied to a hair scalp twice daily with an applicator or is rubbed in with one or more fingers.

In another embodiment the present invention discloses a pharmaceutical foam for stimulating hair growth, preventing hair loss, treating baldness and alopecia in a human subject comprising: a 1-deoxygalactonojirimycin hydrochloride solution in purified water or a buffer, wherein the amount of the 1-deoxygalactonojirimycin hydrochloride is 1 to 15% by weight and one or more inactive ingredients selected from the group consisting of butane, butylated hydroxytoluene, cetyl alcohol, citric acid, glycerin, propylene glycol, polyethylene glycol isobutane, lactic acid, amides, cellulose, vinylpyrrolidone polymers, carboxypolymethylene polysorbate 60, propane, ethyl alcohol, and stearyl alcohol. In one aspect the composition stimulates hair growth, prevents hair loss, treats baldness and alopecia by inhibiting the activity of the one or more scalp enzymes and is applied to a hair scalp twice daily by rubbing into the hair scalp with one or more fingers.

In yet another embodiment the present invention discloses a topical pharmaceutical solution for stimulating hair growth, preventing hair loss, treating baldness and alopecia in a human subject comprising: a 1-15% by weight solution of 1-deoxygalactonojirimycin hydrochloride in purified water, ethyl alcohol, propylene glycol, and a bacteriostatic agent selected from the group consisting of benzoic acid and its esters, benzilic alcohol, and parabens. The topical pharmaceutical solution of the present invention is applied to a hair scalp twice daily with an applicator.

It is contemplated that any embodiment discussed in this specification can be implemented with respect to any method, kit, reagent or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C or combinations thereof” is intended to include at least one of: A, B, C, AB, AC, BC or ABC, and if order is important in a particular context, also BA, CA, CB, CBA, BCA, ACB, BAC or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. H1,551: Topical Composition and Method for Reducing Hair Loss from Human Scalp.

U.S. Pat. No. 6,497,892: Cosmetic and Skin Protective Compositions.

U.S. Pat. No. 7,556,825: Method for Promoting Hair Growth.

¹Schiffmann R. Fabry disease. Pharmacol Ther 2009; 122:65-77.

²Ries M, Moore D F, Robinson C J, et al. Quantitative Dysmorphology Assessment in Fabry Disease. Genet Med 2006; 8:96-101.

³Randall V A. Androgens and Hair Growth. Dermatol Ther 2008; 21:314-28.

What is claimed is:

1. A topical pharmaceutical formulation for stimulating hair growth, preventing hair loss, treating baldness and alopecia in a human subject comprising:
one or more inhibitors of lysosomal α-galactosidase A, wherein the one or more inhibitors are dispersed or dissolved in a continuous phase, wherein the continuous phase is an aqueous phase, an organic phase or an oily phase; and
one or more optional pharmaceutically acceptable excipients, wherein the excipients comprise emollients, thickeners, ionic or non-ionic surfactants and emulsifiers, bacteriostatic agents, coloring agents, scents, and propellant gases or combinations thereof.

2. The formulation of claim 1, wherein the one or more inhibitors of lysosomal α-galactosidase A comprise 1-deoxygalactonojirimycin salts and derivatives, iminosugars and derivatives, deoxyazasugars and derivatives, calystegines alkaloids and derivatives, fagomine isomers, conduritol C epoxides, nortropane alkaloids or combinations thereof.

3. The formulation of claim 1, wherein the inhibitor of the lysosomal α-galactosidase A is 1-deoxygalactonojirimycin hydrochloride.

4. The formulation of claim 1, wherein the 1-deoxygalactonojirimycin hydrochloride is present in an amount of about 1 to 15% by weight in the formulation.

5. The formulation of claim 1, wherein the formulation is applied to a hair scalp once or multiple times in a day.

6. The formulation of claim 1, wherein the formulation stimulates hair growth, prevents hair loss, treats baldness and alopecia by inhibiting the activity of one or more scalp enzymes.

7. The formulation of claim 1, wherein the topical pharmaceutical formulation is a solution, a suspension, an ointment, a foam, an emulsion or a cream.

8. The formulation of claim 1, wherein the emollient comprises glycerin, propylene glycol, polyethylene glycol or combinations thereof.

9. The formulation of claim 1, wherein the thickener comprises amides, cellulose, vinylpyrrolidone polymers, carboxypolymethylene or combinations thereof.

10. The formulation of claim 1, wherein the bacteriostatic agents comprises benzoic acid and its esters, benzilic alcohol, parabens or combinations thereof.

11. The formulation of claim 1, wherein the propellant comprises propane, butane, a mixture of propane and butane or combinations thereof.

12. The formulation of claim 1, wherein the surfactants and emulsifiers comprise laurylsulphate, sorbitane esters and ethers, polyoxymethylene esters and ethers, polyoxyethylene-sorbitane esters, ethers or combinations thereof.

13. The composition of claim 1, wherein the hair loss, the baldness or the alopecia are caused by one or more factors selected from the group consisting of age, genetic make up, environmental effects, chemotherapy, childbirth, fertility drugs, major surgery, poisoning, severe stress, iron deficiency, radiation, mycotic infections, autoimmune disorders, lupus, tumors, skin outgrowths, hypothyroidism, and hyperthyroidism.

14. A method of stimulating hair growth, preventing hair loss or both in a subject comprising the steps of:
identifying a subject in need of stimulation of hair growth, prevention of hair loss or both; and
applying a therapeutically effective amount of a topical pharmaceutical formulation comprising one or more inhibitors of lysosomal α-galactosidase A to a hair scalp of the subject once or multiple times in a day, wherein the one or more inhibitors are dispersed or dissolved in a continuous phase, wherein the continuous phase is an aqueous phase, an organic phase or an oily phase.

15. The method of claim 14, further comprising the step of monitoring the subject for increased hair growth or decreased hair loss following the application of the topical pharmaceutical formulation for a specified period of time.

16. The method of claim 14, wherein the topical pharmaceutical formulation comprises one or more optional pharmaceutically acceptable excipients, wherein the excipients comprise emollients, thickeners, ionic or non-ionic surfactants and emulsifiers, bacteriostatic agents, coloring agents, scents, propellant gases or combinations thereof.

17. The method of claim 14, wherein the topical pharmaceutical formulation is a solution, a suspension, an ointment, a foam, an emulsion or a cream.

18. The method of claim 14, wherein the formulation stimulates hair growth, prevents hair loss or both by inhibiting the activity of one or more scalp enzymes.

19. The method of claim 14, wherein the inhibitor of the lysosomal α-galactosidase A is 1-deoxygalactonojirimycin hydrochloride in an amount of about 1 to 15% by weight in the formulation.

20. A method of treating baldness or alopecia in a subject comprising the steps of:
identifying the subject in need of treatment against the baldness or the alopecia; and
applying a therapeutically effective amount of a topical pharmaceutical formulation comprising one or more inhibitors of lysosomal α-galactosidase A to a hair scalp of the subject once or multiple times in a day, wherein the one or more inhibitors are dispersed or dissolved in a continuous phase, wherein the continuous phase is an aqueous phase, an organic phase or an oily phase.

21. The method of claim 20, further comprising the step of monitoring the subject for hair growth following the application of the topical pharmaceutical formulation for a specified period of time.

22. The method of claim 20, wherein the topical pharmaceutical formulation comprises one or more optional pharmaceutically acceptable excipients, wherein the excipients comprise emollients, thickeners, ionic or non-ionic surfactants and emulsifiers, bacteriostatic agents, coloring agents, scents, propellant gases or any combinations thereof.

23. The method of claim 20, wherein the topical pharmaceutical formulation is a solution, a suspension, an ointment, a foam, an emulsion or a cream.

24. The method of claim 20, wherein the formulation promotes hair growth by inhibiting the activity of the one or more scalp enzymes.

25. The method of claim 20, wherein the alopecia comprises androgenic alopecia, traction alopecia, alopecia areata, alopecia totalis, alopecia universalis, alopecia mucinosa or combinations thereof.

26. The method of claim 20, wherein the inhibitor of the lysosomal α-galactosidase A is 1-deoxygalactonojirimycin hydrochloride.

27. The method of claim 20, wherein the 1-deoxygalactonojirimycin hydrochloride is present in an amount of about 1 to 15% by weight in the formulation.

28. A topical cream or an emulsion for stimulating hair growth, preventing hair loss, treating baldness and alopecia or combinations thereof in a human subject comprising:
a 1-deoxygalactonojirimycin hydrochloride solution in purified water or a buffer, wherein the amount of the 1-deoxygalactonojirimycin hydrochloride is 1 to 15% by weight; and
one or more of the following:
an oil phase selected from the group consisting of solid paraffin, liquid paraffin, silicone oil, squalane, glyceryl monooleate, olive oil, a higher alcohol, a higher aliphatic acid and isopropyl myristate, wherein the oil phase is emulsified with the 1-deoxygalactonojirimycin hydrochloride solution;
an emollient selected from the group consisting of glycerin, propylene glycol, and polyethylene glycol;
a thickener selected from the group consisting of amides, cellulose, vinylpyrrolidone polymers, and carboxypolymethylene;
a surfactant selected from the group consisting of laurylsulphate, sorbitane esters and ethers, polyoxymethylene esters and ethers, polyoxyethylene-sorbitane esters and ethers; and
a bacteriostatic agent selected from the group consisting of benzoic acid and its esters, benzilic alcohol, and parabens.

29. The composition of claim 28, wherein the composition stimulates hair growth, prevents hair loss, treats baldness and alopecia by inhibiting the activity of the one or more scalp enzymes.

30. The composition of claim 28, wherein the composition is applied to a hair scalp twice daily.

31. The composition of claim 28, wherein the composition is applied to the hair scalp with an applicator or is rubbed in with one or more fingers.

32. A pharmaceutical foam for stimulating hair growth, preventing hair loss, treating baldness and alopecia or combinations thereof in a human subject comprising:
a 1-deoxygalactonojirimycin hydrochloride solution in purified water or a buffer, wherein the amount of the 1-deoxygalactonojirimycin hydrochloride is 1 to 15% by weight; and
one or more inactive ingredients selected from the group consisting of butane, butylated hydroxytoluene, cetyl alcohol, citric acid, glycerin, propylene glycol, polyethylene glycol isobutane, lactic acid, amides, cellulose, vinylpyrrolidone polymers, carboxypolymethylene polysorbate 60, propane, ethyl alcohol, and stearyl alcohol.

33. The composition of claim 32, wherein the composition stimulates hair growth, prevents hair loss, treats baldness and alopecia by inhibiting the activity of the one or more scalp enzymes.

34. The composition of claim 32, wherein the composition is applied to a hair scalp twice daily.

35. The composition of claim 32, wherein the composition is rubbed into the hair scalp with one or more fingers.

36. A topical pharmaceutical solution for stimulating hair growth, preventing hair loss, treating baldness and alopecia or combinations thereof in a human subject comprising:
a 1-15% by weight solution of 1-deoxygalactonojirimycin hydrochloride in purified water;
ethyl alcohol;
propylene glycol; and
a bacteriostatic agent selected from the group consisting of benzoic acid and its esters, benzilic alcohol, and parabens.

37. The composition of claim 36, wherein the composition is applied to a hair scalp twice daily.

38. The composition of claim 36, wherein the composition is applied to the hair scalp with an applicator.
